(19) United States
(12) Patent Application Publication
(10) Pub. No.: US 2025/0177627 A1
(43) Pub. Date: Jun. 5, 2025

(54) METHOD AND DEVICE FOR MEASURING OXYGEN SATURATION

(71) Applicant: Vitara Biomedical, Inc., Philadelphia, PA (US)

(72) Inventors: Marcus Graeme Davey, Philadelphia, PA (US); Matthew Slipenchuk, Philadelphia, PA (US); Connor Zabielski, Philadelphia, PA (US); Maddie Tran, Philadelphia, PA (US); Isabel Kuzy, Philadelphia, PA (US); Jake Karwoski, Philadelphia, PA (US); Katie Aleszczyk, Philadelphia, PA (US)

(21) Appl. No.: 18/970,537

(22) Filed: Dec. 5, 2024

Related U.S. Application Data
(60) Provisional application No. 63/606,464, filed on Dec. 5, 2023.

(51) Int. Cl. A61M 1/36; A61M 1/16

(52) U.S. Cl. CPC A61M 1/3666; A61M 1/1601; A61M 1/1698; A61M 1/36224; A61M 2202/0007; A61M 2202/0021; A61M 2202/0208; A61M 2202/0413; A61M 2205/18; A61M 2205/3303; A61M 2205/3313; A61M 2205/3327; A61M 2205/50; A61M 2230/20; A61M 2240/00

(57) ABSTRACT

A sensor system for measuring oxygen saturation in blood flowing within a neonatal extracorporeal support system (NESS) includes a light source configured to emit a light wave, a light sensor configured to sense a light wave, a control unit, and an alarm. The control unit includes a processor operably coupled to at least one memory having instructions stored therein that, upon execution by the control unit, cause the sensor system to perform operations including emitting at least one light wave from the light source onto blood flowing within the NESS, receiving a reflected light wave from the light source onto blood flowing within the NESS, and comparing a parameter of the reflected light wave to a parameter of the at least one light wave to determine the oxygen saturation in the blood of the NESS. The blood flowing within the NESS is unaltered by the sensor system.

### CROSS-REFERENCE TO RELATED APPLICATION

This application claims the benefit of and priority to U.S. Provisional Patent Application Ser. No. 63/606,464, filed on Dec. 5, 2023, the entire content of which is incorporated by reference herein.

### TECHNICAL FIELD

The present disclosure relates generally to neonatal care, and more specifically, devices, systems, and methods for measuring oxygen saturation of a premature fetus outside the womb.

### BACKGROUND

Extreme prematurity is the leading cause of infant morbidity and mortality in the United States, with over one third of all infant deaths and one half of cerebral palsy diagnoses attributed to prematurity. The 2010 Center for Disease Control National Vital Statistics Report notes birth rates at a gestational age of less than 28 weeks in the United States over roughly the past decade have remained stable at approximately 0.7%, or 30,000 births annually. Similarly, birth rates at gestational ages 28-32 weeks over the past decade in the United States have been stable at 1.2%, or 50,000 births annually.

Premature birth may occur due to any one of a multitude of reasons. For example, premature birth may occur spontaneously due to preterm rupture of the membranes (PROM), structural uterine features such as shortened cervix, secondary to traumatic or infectious stimuli, or due to multiple gestation. Preterm labor and delivery is also frequently encountered in the context of fetoscopy or fetal surgery, where instrumentation of the uterus often stimulates uncontrolled labor despite maximal tocolytic therapy.

Respiratory failure represents the most common and challenging problem associated with extreme prematurity, as gas exchange in critically preterm neonates is impaired by structural and functional immaturity of the lungs. Advances in neonatal intensive care have achieved improved survival and pushed the limits of viability of preterm neonates to 22 to 24 weeks gestation, which marks the transition from the canalicular to the saccular phase of lung development. Although survival has become possible, there is still a high rate of chronic lung disease and other complications of organ immaturity, particularly in fetuses born prior to 28 weeks gestation. The development of a system that could support normal fetal growth and organ maturation for even a few weeks could significantly reduce the morbidity and mortality of extreme prematurity and improve quality of life in survivors.

The development of an “artificial placenta” has been the subject of investigation for over 50 years with little success. Previous attempts to achieve adequate oxygen saturation of the fetus in animal models have employed traditional extracorporeal membrane oxygen saturation (ECMO) with pump support and have been limited by circulatory overload and cardiac failure in treated animals. The known systems have suffered from unacceptable complications, including: 1) progressive circulatory failure due to after-load or pre-load imbalance imposed on the fetal heart by oxygenator resistance or by circuits incorporating various pumps; and 2) contamination and fetal sepsis.

There are a variety of methods and devices for measuring oxygen saturation levels in the blood presently on the market. Red blood cells contain hemoglobin molecules through which oxygen binds to the heme on the hemoglobin molecule. These devices typically measure the level of oxygen of arterial, oxygenated blood in the body. It is important to note that fetal hemoglobin levels differ from those of an adult. This is due to the differences in the subunits of hemoglobin between fetus and adults; fetal hemoglobin has a higher affinity for oxygen and will not release oxygen to the tissues as readily.

Oxygen saturation measurement feature is a high-priority for patient care. Pulse oximeters approved by the FDA and released on the market in the last 4 years have a typical SpO₂ accuracy of ±2% (Center for Devices and Radiological Health 2023), and this same error value is also mentioned in the ISO standard document discussing requirements for essential performance of pulse oximetry equipment (British Standards Institution 2023). However, research has conceded that standard pulse oximeter accuracy is lower at readings below 80%, where the deviation between actual and measured saturation can be twice that of the deviation for a reading of blood with saturation at 90% or higher (Schmitt et al. 1993). Accurate readings within both ranges to ensure clinical decisions are accurately made and to reduce the risk of ‘false negative’ measurements. Typical SpO₂ screenings for newborns are successful if the patient has a pulse oximetry reading of 95% or greater (Individual and Family Health n.d.). However, in the first minutes after birth, infants are known to have SpO₂ levels of 50-60%, necessitating accurate readings below 80% (Lara-Cantón et al. 2022).

Typical FDA-approved pulse oximeters are empirically calibrated to relate absorption values to an oxygen saturation percentage, though these initial calibrations are typically performed on healthy subjects breathing air at various oxygen concentrations, only going down to 80%, as here are ethical limitations associated with giving subjects air with lower oxygen concentrations (Nitzan, Romem, and Koppel 2014). This causes issues for patients with hypoxemia, where their SpO₂ results (determined from empirical calibration) may be inaccurate (Zonios, Shankar, and Iyer 2004).

Blood oxygenation can change rapidly, and as such, a continuous output of vital readings is important to keep track of patient health and ensure their safety (Torp et al. 2023). It is necessary not only to take continuous measurements, but also to display these measurements at the same rate.

Accurate SpO₂ measurements are associated with proper clinical decision-making and enhanced patient safety. Exposure of the color sensor to ambient light from the hospital setting has the potential to significantly alter pulse oximetry results (Amar et al. 1989; Yartsev 2020). These issues are especially prevalent in hospital rooms with fluorescent lighting, where the flickering light may present the photodetector with a false pulsatile waveform (typically at a frequency of 50-60 Hz). Besides fluorescent lighting, hospitals utilize quartz-halogen, infrared, incandescent and bilirubin lighting, all of which have the capability to emit light at wavelengths picked up by pulse oximeter photodetectors, and most of which have (anecdotally) been shown to alter SpO₂ readings (Fluck et al. 2003). The prevalence of these lights in clinical settings justifies our requirement that SpO₂ readings shall be accurate within 2% of a control reading at various wavelengths of ambient light, ranging from 200 nm (lower range of bilirubin light peak) to 1000 nm (higher range of incandescent light peak) (Fluck et al. 2003).

Accordingly, a system and method configured to provide extracorporeal support for a premature fetus, or fetuses (preterm or term) with adequate respiratory gas exchange to support life, due to a spectrum of conditions/disorders, may improve viability. A system and method for measuring oxygen saturation may also improve viability.

### SUMMARY

In accordance with an aspect of the disclosure, a sensor system for measuring oxygen saturation in blood flowing within a neonatal extracorporeal support system includes a light source configured to emit a light wave, a light sensor configured to sense a light wave, a control unit, and an alarm operably coupled to the control unit. The control unit includes a processor operably coupled to at least one memory, the memory having instructions stored therein that, when executed by the control unit, cause the sensor system to perform operations including emitting at least one light wave from the light source onto blood flowing within the neonatal extracorporeal support system, receiving a reflected light wave from the blood flowing within the neonatal extracorporeal support system, and comparing a parameter of the reflected light wave to a parameter of the at least one light wave to determine the oxygen saturation in the blood of the neonatal extracorporeal support system, wherein the blood flowing within the neonatal extracorporeal support system is unaltered by the sensor system.

In accordance with another aspect of the disclosure, a method for measuring oxygen saturation in blood flowing within a neonatal extracorporeal support system includes emitting, by a light source of a sensing system, a light wave toward blood flowing within a neonatal extracorporeal support system, wherein the blood flowing within the neonatal extracorporeal support system is unaltered by the sensing system, sensing, by a light sensor of the sensing system, a reflected light wave reflected by the blood flowing within the neonatal extracorporeal support system, comparing, by a control unit operably coupled to the sensing system, a parameter of the reflected light wave to one or more stored values to determine the oxygen saturation in the blood flowing within the neonatal extracorporeal support system, and alerting an alarm of the sensing system if the determined oxygen saturation is outside a predetermined oxygen saturation limit.

### BRIEF DESCRIPTION OF THE DRAWINGS

The foregoing summary, as well as the following detailed description of illustrative embodiments of the application, will be better understood when read in conjunction with the appended drawings. For the purposes of illustrating the present disclosure, there is shown in the drawings illustrative embodiments. It should be understood, however, that the application is not limited to the specific embodiments and methods disclosed, and reference is made to the claims for that purpose. In the drawings:

FIG. 1 is a perspective view of an extracorporeal support system according to an aspect the disclosure;

FIG. 2 is a schematic view of the extracorporeal support system of FIG. 1;

FIG. 3 is a plan view of a portion of the extracorporeal support system of FIG. 1;

FIG. 4 is perspective view of an oxygenator of the extracorporeal support system of FIG. 1;

FIG. 5 is a schematic view of a portion of the extracorporeal support system of FIG. 1, illustrating an oxygenator and a sensor system of an extracorporeal circuit;

FIG. 6 is a schematic view of a controller of the extracorporeal support system of FIG. 1;

FIG. 7 is a perspective, partial cutaway view of a fitting mount of a sensor system of the extracorporeal support system of FIG. 1, showing a venous coupling and an arterial coupling decoupled from the fitting mount;

FIG. 8 is a schematic view of a sensor system of the extracorporeal support system of FIG. 1;

FIG. 9 is a plan view of an arterial coupling attachment fixture of the fitting mount of FIG. 7;

FIG. 10 is an elevation view of the arterial coupling attachment fixture of FIG. 9;

FIG. 11 is a plan view of a venous coupling attachment fixture of the fitting mount of FIG. 7;

FIG. 12 is a front elevation view of the venous coupling attachment fixture of FIG. 11;

FIG. 13 is a side elevation view of the venous coupling attachment fixture of FIG. 11; and

FIG. 14 is a flow diagram of a method of measuring an oxygen saturation of blood flowing within a neonatal extracorporeal support system in accordance with the disclosure.

### DETAILED DESCRIPTION OF ILLUSTRATIVE EMBODIMENTS

Aspects of the disclosure will now be described in detail with reference to the drawings, wherein like reference numbers refer to like elements throughout, unless specified otherwise. Certain terminology is used in the following description for convenience only and is not limiting. The term “plurality”, as used herein, means more than one. The terms “a portion” and “at least a portion” of a structure include the entirety of the structure. Certain features of the disclosure which are described herein in the context of separate embodiments may also be provided in combination in a single embodiment. Conversely, various features of the disclosure that are described in the context of a single embodiment may also be provided separately or in any subcombination.

Described herein is a sensor system that can detect oxygen saturation levels within a fluid, which may be the blood of an animal. In the embodiments described herein, the fluid can be mammalian blood, which may include human blood and the blood of a premature human fetus. The sensor system can detect oxygen saturation levels without contacting the fluid and can utilize visible wavelengths of light to determine the oxygen saturation of a blood sample. The sensor system can detect oxygen saturation levels without utilizing infrared light, and in one particular aspect, the sensor system can determine oxygen saturation in a neonate's or premature fetus's blood in an ex-utero environment.

The sensor system is mountable onto an existing ECMO circuit for measuring arterial and venous saturation in an ECMO circuit that provides accurate blood saturation characterization, and in one particular embodiment, a NICU environment. An example an earlier oximeter device and method is provided in pending PCT/US2023/067310 patent application, published as WO 2023/225684, which is assigned to the Children's Hospital of Philadelphia and incorporated herein by reference in its entirety. This includes accurate readings across varying ambient light environments and oxygen saturation values. In this or other embodiments, the device comprises a calibration mechanism that supports saturation values as low as 30%. In this or other embodiments, the device provides safeguards for patient safety to ensure minimal complications during incubation. In this or other embodiments, the durability of the probes will be increased by not running fiber optics throughout the entire unit. This increases usability by reducing the chance of damaging the probes leading to large expenses or collecting incorrect data. In this or other embodiments, the device comprises a graphical user interface (GUI) that allows the user to view all critical information to aid in clinical decision making. This live trending display would include blood oxygenation values, pre/post oxygenator saturation, and a calibration view.

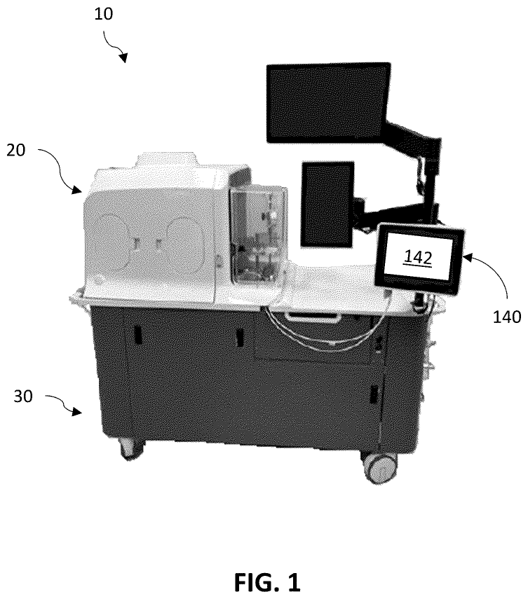

Referring now to the drawings, FIG. 1 illustrates a system for providing extracorporeal support to a patient in accordance with the disclosure and generally identified by reference numeral 10. As can be appreciated, the system 10 can be any extracorporeal support system, and the patient may be any type of patient, human or otherwise, without departing from the scope of the disclosure. In one non-limiting embodiment, the system 10 is configured to provide extracorporeal support to a neonate. According to one aspect of the disclosure, the system 10 is configured to provide a system environment that is similar to an environment the neonate or premature fetus would experience in utero. Viability of a neonate that is removed from the uterine environment (e.g., due to preterm birth) and that is, for example, between about 22 weeks to about 24 weeks gestation, may be increased by placing the neonate in the system 10.

According to an aspect of the disclosure, the system environment may be configured to perform at least one or more of the following: (1) limit exposure of the neonate to light; (2) limit exposure of the neonate to sound; (3) maintain the neonate submerged within a liquid environment; (4) maintain the neonate within a desired temperature range; or (5) any combination thereof. The system 10 also permits neonatal activities (e.g., neonatal breathing movements, neonatal swallowing of fluid) necessary for organ growth and development.

As will be described in further detail hereinbelow, the system disclosed herein is specifically designed to rely on the neonate's heart for blood flow circulation and, accordingly, does not comprise an external mechanical pump, i.e., external to the neonate's heart. Although generally described with respect to neonatal systems, the disclosure is not so limited. It is envisioned that the system 10, or any components thereof, may be used in any extracorporeal system or fluid system without departing from the scope of the disclosure.

The system 10 may be configured to treat neonates (e.g., less than 37 weeks estimated gestational age, particularly 28 to 32 weeks estimated gestational age), and extreme premature neonates (about 23 to 28 weeks estimated gestational age). The gestation periods are provided for humans, though corresponding preterm neonates of other animals may be used. In a particular embodiment, the neonate has no underlying congenital disease. The term or preterm neonate may have limited capacity for pulmonary gas exchange, for example, due to pulmonary hypoplasia or a congenital anomaly affecting lung development, such as congenital disease. In a particular aspect, the subject may be a preterm or a term neonate awaiting lung transplantation, for example, due to congenital pulmonary disease (e.g., bronchoalveolar dysplasia, surfactant protein B deficiency, and the like). Such transplantation surgeries are currently rarely performed in the United States. However, the number of transplantation surgeries may be increased with the more stable method for pulmonary support provided by the instant disclosure. The neonate may also be a candidate for ex utero intrapartum treatment (EXIT) delivery, including patients with severe airway lesions and a long-expected course before definitive resection. The neonate may also be a neonatal surgical or fetoscopic procedure patient, particularly with preterm labor precipitating early delivery. According to one aspect of the disclosure, the system 10 may be configured such that the neonate is maintained in the system 10 for as long as needed (for example, for days, weeks or months, until the neonate is capable of life without the system 10). The system 10 should be operable to maintain the neonate for, without limitation, at least 7 days, at least 14 days, at least 21 days, at least 28 days, at least 35 days, at least 42 days, at least 49 days, or at least 56 days.

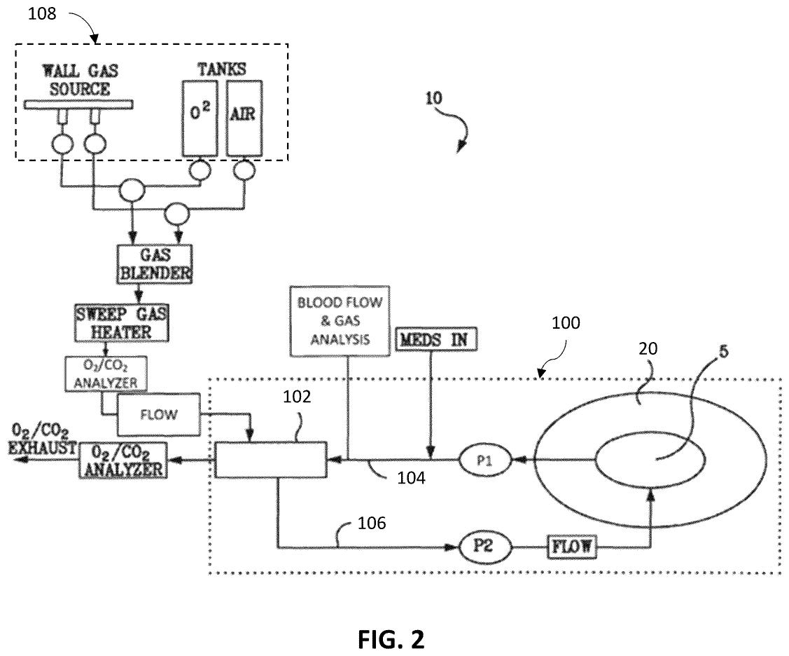

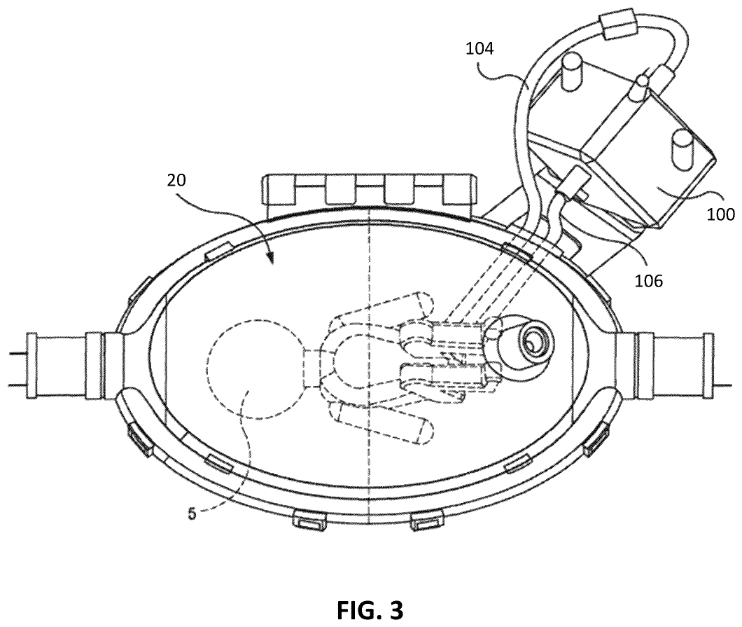

With additional reference to FIGS. 2 and 3, the system 10 includes a neonatal chamber 20 configured to house a neonate 5, a physiologic saline solution (PSS) circuit configured to provide a flow (e.g., a constant flow) of PSS through the neonatal chamber 20, and an oxygenation circuit 200 configured to remove carbon-dioxide from the neonate's 5 blood and supply oxygen to the neonate's 5 blood. The system 10 is configured to maintain the neonate 5 in the neonatal chamber 20 immersed in PSS. The system 10 is further configured such that the oxygenation circuit 100 provides adequate gas exchange for the neonate 5 to sustain life. In this way, the system 10 provides an environment similar to an intrauterine environment to facilitate continued growth and development of the neonate 5. The system 10 may include a cart 30 or other similar device that facilitates monitoring, caring for, and transporting the neonate 5 within a medical facility.

Some non-limiting examples of extracorporeal systems suitable for the treatment of the neonate described herein may be found in the following: U.S. Publ. No. 2021/0052453, now U.S. Pat. No. 11,707,394, titled “Extracorporeal Life Support System and Methods of Use Thereof”; U.S. Publ. No. 2021/0161744, now U.S. Pat. No. 12,083,048, titled “Method And Apparatus For Extracorporeal Support Of Premature Infants”; U.S. Pat. No. 11,471,351, filed on Jun. 13, 2019, and U.S. Publ. No. 2023/0000706 titled “System and Method Configured to Provide Extracorporeal Support for Premature Fetus,” each of which are incorporated herein by reference in their entirety.

The oxygenation circuit 200 may be connected with the neonate 5 in a venous/venous arrangement. Alternatively, the oxygenation circuit 200 may be connected with the neonate 5 in an arterial/venous arrangement. Cannulas may be placed in the great neck vessels (e.g., carotid, jugular) of the neonate 5 to connect the circulatory system of the neonate 5 to one or more oxygenators. The placement in the great neck vessels may avoid issues of vasospasm and cannula instability in umbilical vessels. An external portion of the cannulas may be fitted with a sleeve (e.g., to permit increased tension of the stabilizing sutures). The sleeve may be made of silicone and may be, for example, about 1 to about 10 cm in length, and in embodiments, about 3 to about 5 cm in length. The cannulas may be sutured to the neonate 5 (for example via the fitted sleeve) to secure the cannulas to the neck of the neonate 5.

In some embodiments, the oxygenation circuit 100 may be connected to the neonate 5 via the neonate's 5 umbilical cord. In such an arrangement, cannulas may be sutured into the veins and arteries of the umbilical cord or inserted in the vessels and clamped to the cord. It will be appreciated that other connection arrangements may be utilized. An exemplary non-suturing device is described in U.S. Patent Application Publication No. 2021/0338270, filed on Apr. 28, 2021, the entire content of which is hereby incorporated by reference herein.

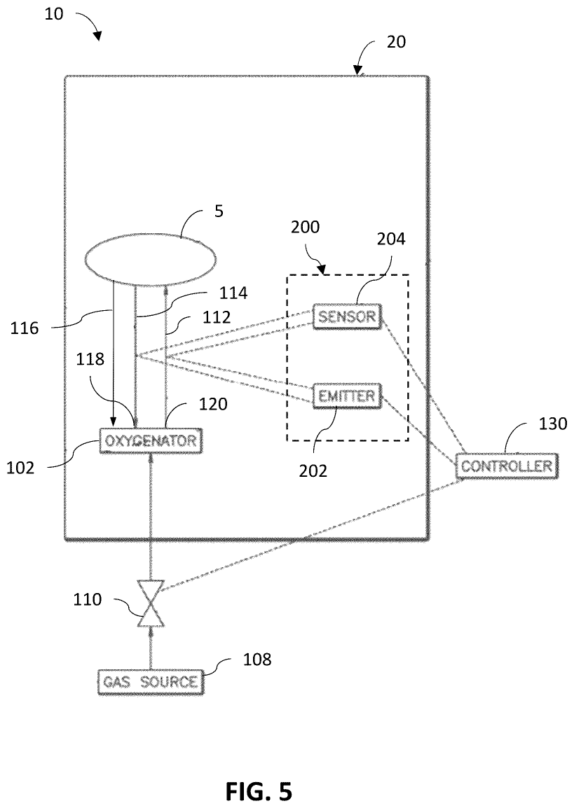

With additional reference to FIGS. 3-5, the oxygenation circuit 100 includes an oxygenator 102. The oxygenator 102 provides gas exchange functionality, particularly of oxygen (to) and carbon dioxide (from), to the neonate 5. It envisioned that the oxygenator 102 may be removably connected to the neonate 5, and optionally, to other components of the oxygenation circuit 100 and/or the system 10. The oxygenation system 100 is connected with the neonate 5 via two or more fluid lines and includes at least a drain line 104 and an inlet line 106. Blood flows from the neonate 5 through the drain line 104 to the oxygenation circuit 100. The blood then flows through the oxygenation circuit 100 and returns to the neonate 5 via the inlet line 106. According to one aspect of the disclosure, before connecting the neonate 5 to the oxygenator 102, the oxygenator 102 may be primed, for example with blood. One or more gasses (e.g., oxygen, carbon dioxide, nitrogen,) can be sent from a gas source 108 to the oxygenator 102 such that the oxygenator 102 introduces oxygen into the blood. A valve 110 (FIG. 5) can regulate the amount of gas supplied from the gas source 108 to the oxygenator 102. The valve 110 can be, for example, a ball valve, control valve, butterfly valve, or globe valve. In some examples, the valve 110 can be adjusted (e.g., moved toward an open position or closed position) in response to receiving an electrical signal. In such an embodiment, the valve 110 is in electrical communication with one or more controllers 130. An exemplary embodiment of oxygenator 106 is shown in pending application PCT US 2022/043509, published as WO 2024/058775, titled “Systems and Methods for Oxygenating Blood, Passive Oxygenation Circuits, and Neonatal Extracorporeal Support Systems,” which is incorporated herein by reference in its entirety.

In an embodiment, cannulating the neonate 5 may include attaching a first cannula 112 to one of a vein and a first artery of the umbilical cord of the neonate 5, and attaching a second cannula 114 to the other of the vein and the first artery of the umbilical cord. In one non-limiting embodiment, cannulating the neonate 5 can include attaching a third cannula 116 (FIG. 5) to a second artery of the umbilical cord of the neonate 5. Cannulating the neonate 5 may further include connecting one or more of the first, second and third cannulae 112, 114, 116 to the oxygenation circuit 100, which includes the oxygenator 102. The first cannula 112 can be in fluid communication with the drain line 104 and/or an inlet 118 of the oxygenator 102. The second cannula 114 can be in fluid communication with the inlet line 106 and/or an outlet 120 of the oxygenator 102. One non-limiting example of a cannula contemplated for use is disclosed in U.S. Patent Application Publication Number 2021/0338270, titled “Cannula Insertion System and Methods of Using the Same,” filed on Apr. 28, 2021, which is incorporated herein by reference in its entirety.

Each of the first and second cannulae 112, 114 can include a first end and a second end opposite the first end in a first direction. The first and second ends of the first cannula 112 can be fluidly coupled to the neonate 5 and the inlet 118 of the oxygenator 102, respectively. The first and second ends of the second cannula 114 can be fluidly coupled to the neonate 5 and the outlet 120 of the oxygenator 102, respectively. Each of the first and second cannulae 112, 114 can include first and second sides opposite each other in a second direction perpendicular to the first direction.

A sensor system 200 can measure oxygen saturation in the blood of the neonate 5. The sensor system 200 can be and/or include an oximeter that utilizes visible wavelengths of light to determine the oxygen saturation of a blood sample. The sensor system 200 can determine oxygen saturation without directly contacting a blood sample, lowering the risk of infection and patient disruption as compared to determining oxygen saturation by directly contacting the blood sample. In some examples, the sensor system 200 includes a transmissive oximeter. In other examples, the sensor system 200 includes a reflective oximeter. In transmissive oximetry, a sensor (e.g., photodiode) and a light source are positioned on opposite sides of a measurement site. The light is emitted from the light source, transmitted through the measurement site, and received by the sensor. In reflective oximetry, both the sensor and the light source are on the same side of the measurement site. The light is emitted from the light source, reflected by the blood, and received by the sensor. Reflective oximetry can be utilized for measurement sites having increased depth and/or density compared to transmissive oximetry sites.

The sensor system 200 can include a light source or emitter 202 and a sensor 204. The emitter 202 can emit a wave (e.g., a light wave) toward at least one of the first and second cannulae 112, 114. The sensor 204 can detect a reflected portion of the wave. The emitter 202 and the sensor 204 can each be positioned on the same side of the first and second cannulae 112, 114, although other configurations are contemplated without departing from the scope of the disclosure. The emitter 202 and the sensor 204 can each be positioned on the same side of the oxygenator 102, although other configurations are contemplated without departing from the scope of the disclosure. The sensor system 200 can use reflective oximetry when the emitter 202 and the sensor 204 are each positioned on the same side of the oxygenator 102.

The emitter 202 can be a light source that emits light configured to be reflected and sensed by the sensor 204. The emitter 202 can, for example, be a light emitting diode (“LED”) array, can include a plurality of light sources, and can emit white light. The emitter 202 can emit, for example, light having a wavelength ranging from about 400 nanometers to about 1 millimeter, about 400 nanometers to about 800 nanometers, about 700 nanometers to about 1 millimeter, about 450 nanometers to about 600 nanometers, about 400 nanometers to about 500 nanometers, or about 600 nanometers to about 800 nanometers. In embodiments, the emitter 202 can emit one or more of red light, green light, blue light, and infrared light. In one non-limiting embodiment, the emitter 202 can emit a combination of light wavelengths, such as, for example, a combination of red, green, and blue lights to provide a white light. The emitter 202 can emit a first light wave having a wavelength of about 625 nanometers to about 775 nanometers, a second light wave having a wavelength of about 475 nanometers to about 600 nanometers, a third light wave having a wavelength of about 400 nanometers to about 500 nanometers. In embodiments, the emitter 202 can include a plurality of LEDs and/or an array of LEDs. In one particular embodiment, the emitter 202 can include four LEDs arranged in an array.

The sensor 204 can be configured to sense one or more light waves, which in embodiments, can be reflected light waves. The sensor 204 can be configured to sense light waves emitted by the emitter 202 and reflected by blood of the neonate 5. The sensor 204 can be configured to sense a plurality of light waves at different wavelengths. The sensor 204 can be configured to simultaneously sense a plurality of light waves at different wavelengths. The sensor 204 can be configured to sense a first light having a wavelength of about 625 nanometers to about 775 nanometers, a second light wave having a wavelength of about 475 nanometers to about 600 nanometers, and/or a third light wave having a wavelength of about 400 nanometers to about 500 nanometers. The sensor 204 can be configured to sense, for example, one or more of red light, blue light, and/or green light. Some sensors contemplated for use in the sensor system 200 are, without limitation, the AS7341 Spectral Sensor from Adafruit Industries LLC and the TCS230 sensor.

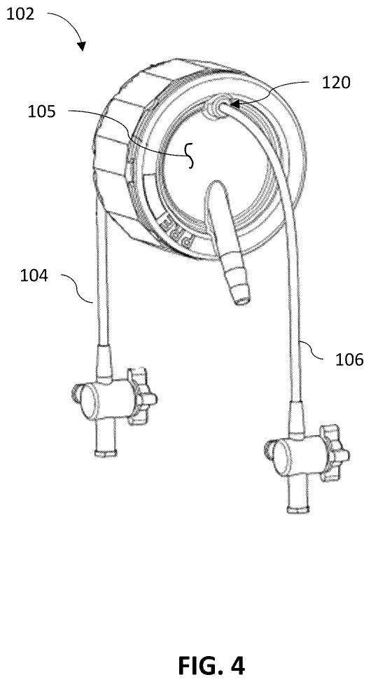

The sensor system 200 can be configured to determine the oxygen saturation of blood within the oxygenator 102. Referring again to FIG. 4, the oxygenator 102 can include a transparent face 105 such that light can pass through the face 105. The emitter 202 can be positioned adjacent the face 105 and emit a light wave through the transparent face 105 such that light is reflected off the blood within the oxygenator 102. The sensor 204 can be positioned adjacent the face 105 so as to sense the light reflected from the blood within the oxygenator 102. In some examples, at least one of the sensor 204 and the emitter 202 are coupled to the oxygenator 102. In some examples, at least one of the sensor 204 and the emitter 202 are fixed to the oxygenator 102. The sensor 204 and the emitter 202 can be fixed to the transparent face 105 of the oxygenator 102.

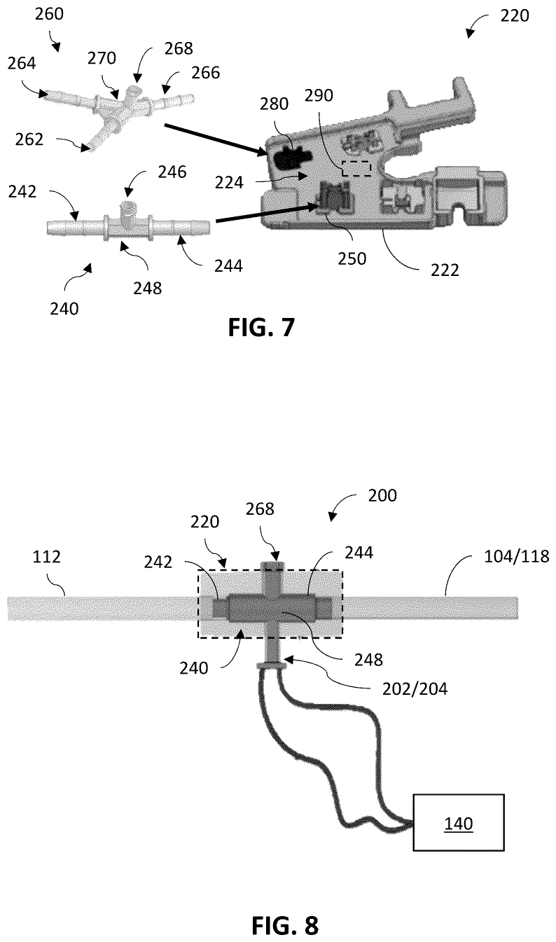

Turning to FIGS. 7-13, a fitting mount of the sensor system 200 is illustrated and generally identified by reference numeral 220. The fitting mount 220 includes a housing 222 in which at least a portion of each of the emitter 202 and the sensor 204 is disposed. The housing 222 defines an inner cavity 224 for receipt of at least one of a venous coupling 240 and an arterial coupling 260. Although generally illustrated as being a tee coupling, it is envisioned that the venous coupling 240 may define any suitable configuration without departing from the scope of the disclosure. The venous coupling 240 and the arterial coupling 260 are couplable to any suitable tubing for use in the medical field and with the system 10 described herein. In this manner, the sensor system 200 measures oxygen concentration of the blood flowing through the system 10 without interacting or otherwise interfering with the blood.

In the example embodiment, the venous coupling 240 defines an inlet portion 242, an outlet portion 244, and a port 246 interposed between and in fluid communication with, each of the inlet portion 242 and the outlet portion 244. In one non-limiting embodiment, the inlet portion 242 is selectively couplable to the first cannula 112 and the outlet portion 244 is selectively couplable to the drain line 104 and/or the inlet 118 of the oxygenator 102 to transfer blood from the neonate 5 through the venous coupling 240 and to the oxygenator 102. The port 246 is selectively couplable to another source of fluids and/or medicaments, such as a syringe, an intravenous bag, etc. In the example embodiment, a center portion 248 of the venous coupling 240 defines a generally hexagonal or multifaceted profile extending in a longitudinal direction through each of the inlet portion 242 and the outlet portion 244, although the center portion 248 may define any suitable profile without departing from the scope of the disclosure. As will be described in further detail hereinbelow, the hexagonal profile of the center portion 258 is selectively couplable to a venous coupling attachment fixture 250 to selectively couple the venous coupling 240 to the housing 222 and provides a flat surface area through which the emitter 202 can emit light waves and the sensor 204 can receive waves reflected by the blood flowing through the venous coupling 240.

Although generally illustrated as defining a Y-shaped profile, it is envisioned that the arterial coupling 260 may define any suitable profile without departing from the scope of the disclosure. In the example embodiment, the arterial coupling 260 defines a first inlet portion 262, a second inlet portion 264, an outlet portion 266, and a port 268. In embodiments where only one cannula is connected to an artery of the umbilical cord, the arterial coupling 260 may define a tee shaped profile or one of the first inlet portion 262 or the second inlet portion 264 may be plugged or capped to inhibit blood from flowing out of the arterial coupling 260. In the exemplary embodiment, the first inlet portion 262 may be selectively coupled to one of the second cannula 114 and third cannula 116 and the second inlet portion 264 may be selectively coupled to the other one of the second cannular 114 or the third cannula 116. The outlet port 266 is selectively couplable to the inlet line 106 and/or the outlet 120 of the oxygenator 102 to return blood to the neonatal 5 from the oxygenator 102. The port 268 is interposed between, and in fluid communication with, the first inlet portion 262, the second inlet portion 264, and outlet portion 266. The port 268 may be selectively coupled to a syringe, bag, or any other component for receiving blood. The arterial coupling 260 defines a center portion 270 having a generally hexagonal or multifaceted profile extending in a longitudinal direction through each of the first inlet portion 262, the second inlet portion 264, and the outlet portion 266 and intersecting at a generally midportion of the arterial coupling 260. It is envisioned that the center portion 270 of the arterial coupling 260 may define any profile and each of the first inlet portion 262, the second inlet portion 264, and the third inlet portion 266 may include the same or different profile. The hexagonal profile of the center portion 270 of the arterial coupling 260 is selectively couplable to an arterial coupling attachment fixture 280 to selectively couple the arterial coupling 260 to the housing 222.

In one embodiment, the sensor system 200 described herein includes LEDs 290 that emit light on the blood to reflect back onto the sensor 204 that corrects for exposure to ambient light. The LEDs 290 may be programmed to run at various frequencies (potentially to match the operating room's (OR) and/or neonatal intensive care unit's (NICU) main lighting), and blood oxygen saturation readings may be, for example, accurate within about ±2% on either side of a measurement of blood when exposed to various light sources present within a hospital setting, and in embodiments, may comply with ISO 201.12.1.101.1-2 SO2 accuracy of pulse oximeter equipment. It is envisioned that the housing 222 may be formed from any suitable material, and may be formed from an opaque, translucent, or transparent material without departing from the scope of the disclosure.

With reference to FIG. 8, a schematic diagram of the sensor system 200 is illustrated and shown coupled to the first cannula 112 and the drain line 104 and/or the inlet 118 of the oxygenator 102. In the example embodiment, the emitter 202 and the sensor 204 are integrated with or form part of a camera or other suitable imaging device that is operably coupled to the venous coupling 240 and the controller 140. The venous coupling attachment fixture 250 selectively couples the venous coupling 240 to the housing 222 and enables the emitter 202 and the sensor 204 to emit light waves and receive light waves reflected by blood flowing through the venous coupling 240. The hexagonal profile of the center portion 248 provides a generally planar surface through which the light waves can travel, minimizing interference, refraction, and reflection of the light waves propagating through the venous coupling 240. In one non-limiting embodiment, the venous coupling attachment fixture 250 spaces the venous coupling 240 from the emitter 202 and the sensor 204 by about 30 mm. As can be appreciated, the venous coupling attachment fixture 250 provides stability and maintains a position of the venous coupling 240 relative to the emitter 202 and the sensor 204.

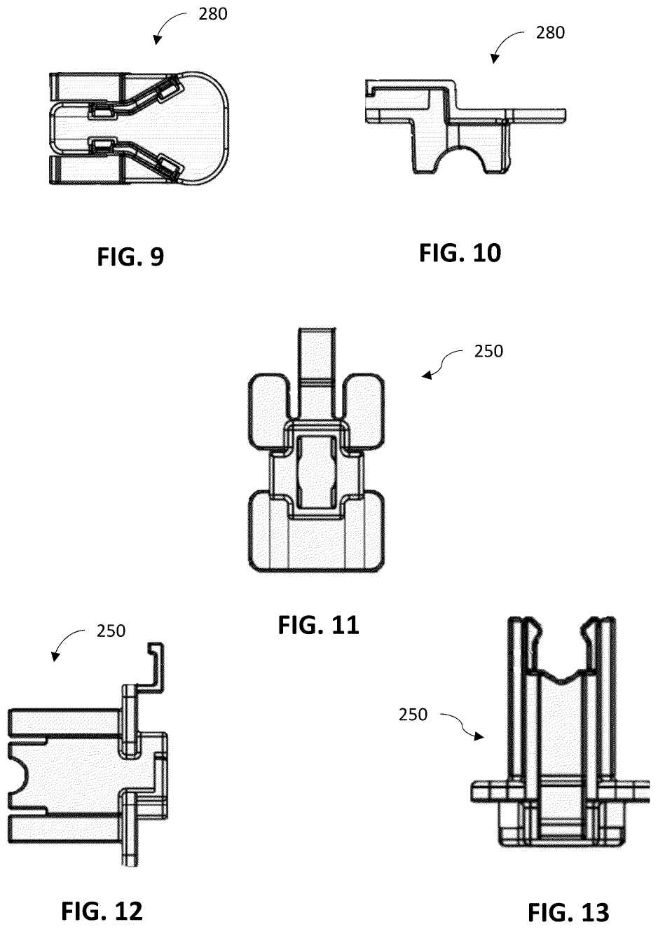

FIGS. 9-13 illustrate different views and features of each of the venous coupling attachment fixture 250 and the arterial coupling attachment fixture 280. In one non-limiting embodiment, the venous coupling attachment fixture 250 may include one or more features for retaining the emitter 202 and/or the sensor 204 and aid with maintaining the position of the venous coupling 240 relative to the emitter 202 and/or the sensor 204.

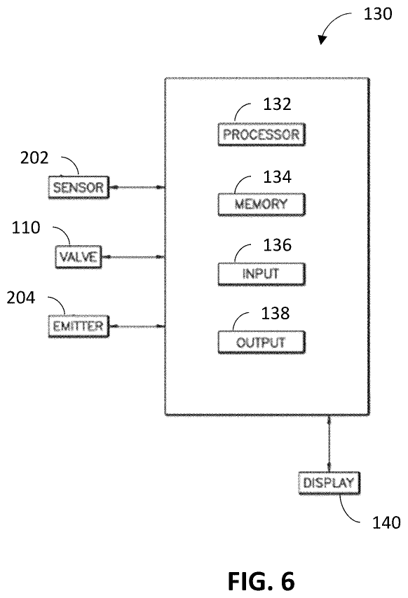

Returning to FIGS. 5 and 6, the controller 130 is configured to send and receive electrical signals. For example, the controller 130 can send and receive signals from at least one of the sensor 204 and the emitter 202. The controller 130 can send an emitter signal so as to cause the emitter 202 to emit the light wave. The emitter signal can include a parameter of the emitted light wave, which may be, for example, the length of time the light wave is emitted, the intensity of the light wave emitted, the wavelength at which the light wave is emitted, the number of light waves emitted, and/or the number of lumens emitted. The amount of light emitted can also be referred to as the number of lumens emitted.

The sensor 204 can send a sensor signal to the controller 130. In some examples, the sensor 204 sends the sensor signal directly to the controller 130. In other examples, the sensor 204 sends the sensor signal to one or more intermediate components (not shown) that send a signal to the controller 130 in response to receiving the sensor signal. The sensor signal can include one or more parameters indicative of a property of the reflected light wave, such as, for example, a parameter indicative of the amount of reflected light sensed by the sensor 204, a parameter indicative of the number of lumens of reflected light sensed by the sensor 204, a parameter indicative of the number of wavelengths sensed by the sensor 204, a parameter indicative of the wavelength of each light wave sensed by the sensor 204, and/or a parameter indicative of the length of time over which the light wave was sensed. In one non-limiting embodiment, the sensor signal can indicate the amount of each light wavelength sensed by the sensor 204.

The wavelength of the reflected light sensed by the sensor 204 can be indicative of the oxygen saturation. The sensor 204 can sense red, green, and blue wavelengths within the reflected light. The sensor signal can include the number of lumens for each of the red, green, and blue wavelengths, where the number of lumens for the red, green, and blue wavelengths can be indicative of the oxygen saturation.

The controller 130 can include a processor 132 and at least one memory 134. The at least one memory 134 can have instructions stored therein that cause the sensor system 200 to perform operations including emitting at least one light wave from the emitter 202, receiving a reflected light wave with the sensor 204, and comparing a parameter of the reflected light wave to one or more stored values to determine the oxygen saturation of the blood of the neonate 5. The controller 130 can include an input 136 that receives the sensor signal from the sensor 204. The controller 130 can include an output 138 that sends an electrical signal. For example, the output 138 can send the emitter signal to the emitter 202. The controller 130 can store a parameter of the emitted light wave and/or a parameter of the reflected light wave in the at least one memory 134.

The processor 132 can compare the parameters of the emitted and reflected light waves to each other to determine the amount of light absorbed by the blood of the neonate 5. The amount of light absorbed by the blood of the neonate 5 can be determined from the amount of light emitted by the emitter 202 compared to the amount of reflected light sensed by the sensor 204. The at least one memory 134 can include a table of values indicative of oxygen saturation in blood. The processor 132 can compare the amount of light absorbed by the blood of the neonate 5 to the table values so as to determine the oxygen saturation of the blood of the neonate 5. The processor 132 can compare the number of lumens of each of the red, green, and blue wavelengths sensed by the sensor 204 to the table values. In some examples, the table values can be created by comparing different levels of oxygen saturation determined by the sensor system 200 to corresponding levels of oxygen saturation as determined by other existing systems. The processor 132 can compare the oxygen saturation of the blood to a threshold. The sensor system 200 can determine oxygen saturation levels ranging from about 30% to about 100%, about 30% to about 45%, about 45% to about 60%, about 60% to about 75%, about 75% to about 90%, or about 90% to about 100% in the blood of the neonate 5. In some embodiments, the sensor system 200 can sense the oxygen saturation of the neonate's 5 blood at the inlet 118 and outlet 120 of the oxygenator 102. The processor 132 can determine the amount of oxygen absorbed by the neonate 5 by comparing the oxygen saturation of the blood at the inlet 118 and outlet 120 of the oxygenator 102.

The controller 130 can send a valve signal so as to adjust an amount of gas supplied from the gas source 108 to the oxygenator 102. In some examples, the controller 130 sends the valve signal directly to the valve 110. In other examples, the controller 130 sends the valve signal to one or more intermediate components (not shown) that send a signal to the valve 110 in response to receiving the valve signal. The controller 130 can send the valve signal to increase the gas supplied to the oxygenator 102 if the oxygen saturation is below the threshold and the controller 130 can send the valve signal to decrease the gas supplied to the oxygenator 102 if the oxygen saturation is above the threshold.

The sensor system 200 can include or otherwise be operably coupled to a display 140 configured to display information regarding the oxygen saturation of the blood of the neonate 5. The controller 130 can send a display signal so as to cause the display 140 to display the information. In some examples, the controller 130 sends the display signal directly to the display 140. In other examples, the controller 130 sends the display signal to one or more intermediate components (not shown) that send a signal to the display 140 in response to receiving the display signal. The display 140 can be a monitor, television, or other electronic display. In some embodiments, the controller 130 can send the display signal to a computer (not shown). In some examples, the controller 130 can store the oxygen saturation levels in the at least one memory 134. It is envisioned that the display 140 may be integrated with the sensor system 200 (e.g., disposed on or within the oxygenator 102, etc.) or may be a component of the system 10, which may be mounted on any portion of the system 10, such as, for example, the cart 30 (FIG. 1).

In embodiments, the sensor system 200 may emit a first light wave and a second light wave having a different wavelength than the first light wave. For example, the first light wave can be a red light wave having a wavelength of approximately 660 nm and the second light wave can be an infrared light having a wavelength of approximately 940 nm. The light waves can be emitted sequentially such that only one of the first and second light waves are emitted at a time. However, this can require multiple light sources and the sequential emission can increase the time necessary to determine oxygen saturation levels. In the example embodiment, the sensor system 200 can emit a light from a single light source or emitter 202 and the sensor 204 can sense multiple wavelengths simultaneously.

In one embodiment, the sensor system 200 measures SpO₂ levels relatively quickly and accurately. In one embodiment of the sensor system 200 described herein, the sensor system 200 measures SpO₂ at a quick sampling rate, with accuracy to within 2% on either side of the SpO₂ value from a pre-existing validated oximeter. To safeguard from extended periods of inaccurate readings, the sensor system 200 may allow calibration to recalibrate its readings against a validated oximeter. In this or other embodiments, the sensor system 200 includes an alarm to notify clinicians when no data has been reported to the sensor system 200 for more than 30 seconds or other time interval.

In another embodiment, the sensor system 200 described herein includes a GUI 142 to provide users, such as health care providers, current and past SpO₂ readings or a patient, as well as provide a tab for measurement calibration. The GUI 142 contains tabs that provide various language options as well as universal units. In one non-limiting embodiment, the sensor system 200 uses scientific units.

In another embodiment, the sensor system 200 is used on a pre-existing external blood circuit, where venous blood exits the patient, passes through an oxygenator and returns into the patient's artery. Thus, the light source or emitter 202, detector or sensor 204, and their fitting mount 220 are able to connect to the circuit without impeding steady flow through its tubing.

Oxygenation of a patient's venous blood for return into their artery requires vessel cannulation and once the patient is healthy enough to be removed from the care of this system 10, decannulation is required. This causes periods where the patient's blood supply is disconnected from the oxygenation circuit 100, which may include the risk of blood spraying from the patient's umbilical cord and splashing on one or more components of the sensor system 200. Blood splashing onto the emitter 202 and/or the sensor 204 may damage circuitry and alter color detection results. As such, the emitter 202 and/or the sensor 204 must be able to be cleaned after each incubation trial. Consequently, in one embodiment of the sensor system 200 described herein, the sensor system 200 can be cleaned without product degradation. In this or other embodiments, the sensor system 200 passes the Ingress Protection Grade 2 (IPX2), i.e., protected against spraying water when tilted up to 15 degrees vertically.

In the example embodiment, the sensor system 200 retains a temperature range that does not negatively affect the patient's blood as it travels through the oxygenation circuit 100. In one non-limiting embodiment, the sensory system 200 is at a temperature of about 41 degrees Celsius or below to prevent the blood from coagulating.

In embodiments, the sensor system 200 includes a microcontroller associated with the sensor system's 200 emitter 202 and/or sensor 204 and stores measured various wavelength data and base calibration data in the memory 134. In this or other embodiments, the data may be plotted and shown on the GUI display 142 to show data trends. In embodiments, the sensor system 200 may retain patient data for up to twelve hour intervals before being overwritten or alternatively transferred to cloud storage (not shown).

A typical health range for SpO₂ for a fetal or neonatal patient ranges from about 90% to about 98% SO₂. In embodiments, the sensor system 200 includes an alarm that sounds if SpO₂ readings of about 30% or below, about 89% or below, and/or about 98% or above are reported for fetal or neonatal patients. The alarm feature on the sensor system 200 may be programmable to accommodate various alarm settings. For example, the alarm can sound if no SpO₂ data is reported to the data display 130 for greater than 30 seconds or any other time interval. Additionally, fluctuations in SpO₂ levels about a certain preset range can also be documented and alarmed. In embodiments, an alarm for a lack of data received when the sensor system 200 is improperly connected to the oxygenation circuit 100 can be used to minimize instances where the sensor system 200 is improperly connected to the oxygenation circuit 100.

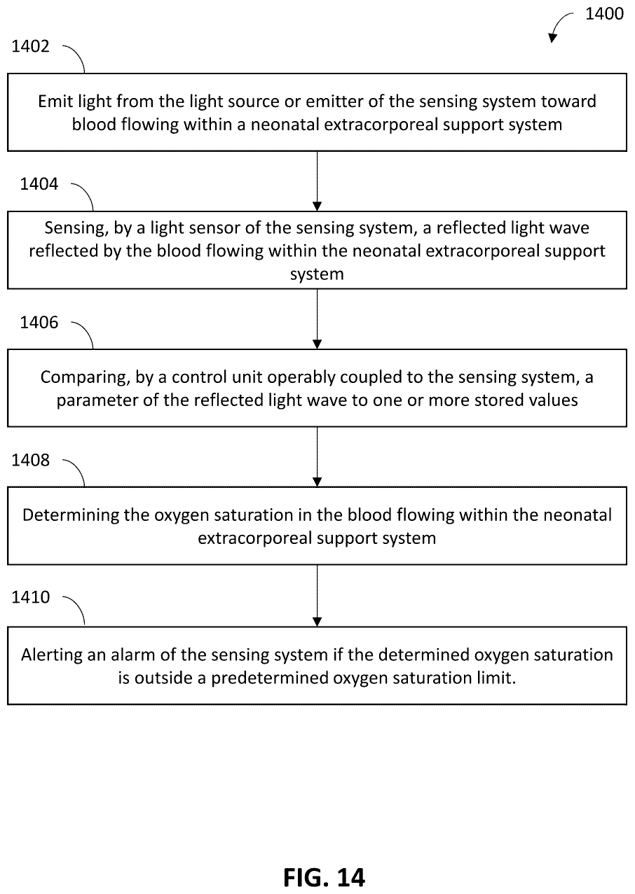

Turning to FIG. 14, a method of measuring an oxygen saturation of blood flowing within a neonatal extracorporeal support system is illustrated and generally identified by reference numeral 1400. A light wave is emitted 1402 from the light source or emitter of the sensing system toward blood flowing within a neonatal extracorporeal support system. The blood flowing within the neonatal extracorporeal support system is unaltered by the sensing system. A light wave that is reflected by the blood flowing within the neonatal extracorporeal support system is sensed 1404 by a light sensor of the sensing system. A parameter of the reflected light wave is compared 1406, by the controller or control unit operably coupled to the sensing system, to one or more stored values to determine 1408 the oxygen saturation in the blood flowing within the neonatal extracorporeal support system. If the determined oxygen saturation is outside a predetermined oxygen saturation limit, an alarm system of the sensing system is alerted 1410.

It will be appreciated that the foregoing description provides examples of the disclosed system and methods. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

Recitation of ranges of values herein are merely intended to serve as a shorthand method of referring individually to each separate value falling within the range including the stated ends of the range, unless otherwise indicated herein, and each separate value is incorporated into the specification as if it were individually recited herein. All methods described herein can be performed in any suitable order unless otherwise indicated herein or otherwise clearly contradicted by context. The term “about” as used herein in reference to numerical ranges can mean the stated value +/−1%, 2%, 3%, 4%, 5% or 10%.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. As one of ordinary skill in the art will readily appreciate from the disclosure of the present invention, processes, machines, manufacture, composition of matter, means, methods, or steps, presently existing or later to be developed that perform substantially the same function or achieve substantially the same result as the corresponding embodiments described herein may be utilized according to the present disclosure.

What is claimed is:

1. A sensor system for measuring oxygen saturation in blood flowing within a neonatal extracorporeal support system, the sensor system comprising:
a light source configured to emit a light wave;
a light sensor configured to sense a light wave;
a control unit, wherein the control unit includes a processor operably coupled to at least one memory, the memory having instructions stored therein that, upon execution by the control unit, cause the sensor system to perform operations comprising:
emitting at least one light wave from the light source onto blood flowing within a neonatal extracorporeal support system;
receiving a reflected light wave from the blood flowing within the neonatal extracorporeal support system with the light sensor; and
comparing a parameter of the reflected light wave to a parameter of the at least one light wave to determine the oxygen saturation in the blood of the neonatal extracorporeal support system; and
an alarm operably coupled to the control unit,
wherein the blood flowing within the neonatal extracorporeal support system is unaltered by the sensor system.

2. The sensor system of claim 1, further comprising an oxygenator in fluid communication with the blood of the neonatal extracorporeal support system, the oxygenator including a transparent face, wherein the light source is configured to emit a light wave through the transparent face and the light sensor is configured to sense a light wave through the transparent face,
wherein the memory includes further instructions stored therein that, upon execution by the control unit, cause the sensor system to perform the operation comprising determining the oxygen saturation of the blood within the neonatal extracorporeal support system.

3. The sensor system of claim 2, wherein the oxygenator introduces oxygen into the blood of the neonatal extracorporeal support system, and
wherein the memory includes further instructions stored therein that, upon execution by the control unit, modifies an amount of oxygen supplied to the oxygenator to modify an amount of oxygen introduced into the blood of the neonatal extracorporeal support system by the oxygenator.

4. The sensor system of claim 1, wherein the light source emits a first light wave at a first wavelength and emits a second light wave at a second wavelength different from the first wavelength,
wherein the light source emits the first light wave and the second light wave through at least one of a venous coupling or an arterial coupling of the neonatal extracorporeal support system.

5. The sensor system of claim 4, wherein the light source emits a third light wave at a third wavelength that is different from each of the first wavelength of the first light wave and the second wavelength of the second light wave.

6. The sensor system of claim 1, wherein the at least one light wave is at a wavelength ranging from about 400 nanometers to about 700 nanometers.

7. The sensor system of claim 6, wherein the light sensor senses a first reflected light wavelength and a second reflected light wavelength different from the first wavelength.

8. The sensor system of claim 7, wherein the light sensor senses a third reflected light wavelength that is different from each of the first reflected light wavelength and the second reflected light wavelength.

9. The sensor system of claim 7, wherein the light sensor senses the first and second reflected light wavelengths in response to the light source emitting a single light wave at a selected wavelength.

10. The sensor system of claim 7, wherein the light sensor senses the first and second reflected light wavelengths simultaneously.

11. The sensor system of claim 1, wherein the at least one memory has instructions stored therein that, upon execution by the control unit, causes the sensor system to perform operations comprising:
comparing a parameter of the reflected light wave to one or more stored values to determine the oxygen saturation in the blood of the neonatal extracorporeal support system.

12. The sensor system of claim 1, wherein the sensor system senses oxygen saturation levels ranging from about 30% to about 100%.

13. The sensor system of claim 12, wherein the sensor system senses oxygen saturation levels ranging from about 40% to about 75%.

14. A method for measuring oxygen saturation in blood flowing within a neonatal extracorporeal support system, the method comprising the steps of:
emitting, by a light source of a sensing system, a light wave toward blood flowing within a neonatal extracorporeal support system, wherein the blood flowing within the neonatal extracorporeal support system is unaltered by the sensing system;
sensing, by a light sensor of the sensing system, a reflected light wave reflected by the blood flowing within the neonatal extracorporeal support system;
comparing, by a control unit operably coupled to the sensing system, a parameter of the reflected light wave to one or more stored values to determine the oxygen saturation in the blood flowing within the neonatal extracorporeal support system; and
alerting an alarm of the sensing system if the determined oxygen saturation is outside a predetermined oxygen saturation limit.

15. The method of claim 14, wherein emitting the light wave includes emitting a light wave at a wavelength of about 400 nanometers to about 700 nanometers,
wherein the light wave is emitted through at least one of a venous coupling or an arterial coupling of the neonatal extracorporeal support system.

16. The method of claim 14, wherein the method further comprises determining the oxygen saturation in the blood of the neonatal extracorporeal support system flowing within an oxygenator having a transparent face, wherein the light wave is emitted through the transparent face and the reflected light wave is sensed by the light sensor through the transparent face.

17. The method of claim 14, wherein the method further comprises determining the oxygen saturation in the blood of the neonatal extracorporeal support system without infrared light.

18. The method of claim 17, wherein determining the oxygen saturation in the blood of the neonatal extracorporeal support system includes each of the light source and the light sensor positioned on a same side of at least one of an oxygenator or a venous coupling of the neonatal extracorporeal support system.

19. The method of claim 14, wherein the method further comprises modifying, by the control unit, an amount of oxygen supplied to an oxygenator of the neonatal extracorporeal support system to modify the amount of oxygen introduced into the blood of the neonatal extracorporeal support system by the oxygenator.

20. The method of claim 14, wherein the sensing step includes sensing, by the light sensor, a first reflected light wave at a first wavelength, a second reflected light wave at a second wavelength different from the first wavelength, and a third reflected light wave at a third wavelength different from each of the first reflected light wavelength and the second reflected light wavelength, wherein each of the first, second, and third light waves are a reflection of the light wave emitted by the light source
